(19) United States
(12) Patent Application Publication
(10) Pub. No.: US 2021/0113077 A1
(43) Pub. Date: Apr. 22, 2021

(54) NEW TYPE OF LARYNGOSCOPE TUBE CORE

(71) Applicant: JIANGXI SAI XIN MEDICAL TECHNOLOGY CO., LTD, Nanchang (CN)

(72) Inventor: Yaozan WANG, Nanchang (CN)

(73) Assignee: JIANGXI SAI XIN MEDICAL TECHNOLOGY CO., LTD, Nanchang (CN)

(21) Appl. No.: 17/120,639

(22) Filed: Dec. 14, 2020

Related U.S. Application Data
(63) Continuation of application No. PCT/CN2019/083439, filed on Apr. 19, 2019.

(30) Foreign Application Priority Data
Jul. 13, 2018 (CN) 2018107717780

(51) Int. Cl. A61B 1/267; A61B 1/12; A61B 1/00

(52) U.S. Cl. CPC A61B 1/267; A61B 1/00114; A61B 1/00004; A61B 1/127

(57) ABSTRACT

A new type of laryngoscope tube core is disclosed. The laryngoscope tube core includes a smart connector shell (1), a tube core (4) and an endoscope (6) arranged at the end of the tube core (4). The smart connector shell (1) includes an upper cover (10) and a lower cover (11) and the smart connector shell (1) is provided with a circuit board (3). The circuit board (3) is provided with a single-chip microcomputer (302). One end of the smart connector shell (1) is provided with a MINI USB plug (301), and the MINI USB plug (301) passes through the smart connector shell (1) and is connected to the circuit board (3). The tube core (4) is inserted into and fixed to the smart connector shell (1), and the end of the tube core (4) inserted into the smart connector shell (1) is connected to the circuit board (3).

### TECHNICAL FIELD

The present disclosure relates to the field of laryngoscopes, and more specifically, to a new type of laryngoscope tube core.

### BACKGROUND

Due to the deep position of the larynx and the complex physiological structure, it cannot be directly seen. Some special inspection methods are required for laryngoscopy, such as indirect laryngoscope, direct laryngoscope, fiber laryngoscope, electronic laryngoscope, strobe laryngoscope, ultra-high-speed cinematography, sonogram or glottograph.

The electronic laryngoscope is generally composed of a camera, a hollow hard tube (or “connecting rod”), a handle and a control display module; the front end of the hollow hard tube is equipped with a camera, and the rear end is fixedly connected with the handle, and a wire can pass through it; the rear of the handle is connected with a control display module. In the process of diagnosis and treatment using the electronic laryngoscope, the electronic laryngoscope is generally used with a tracheal tube (or “tracheal intubation”, or “insertion tube”). The hollow hard tube of the electronic laryngoscope is inserted into the tracheal tube, and the tracheal tube is circumferentially covered with the hollow hard tube with the camera and penetrates into the patient's throat cavity. Because the structure of the connection between the laryngoscope and the connecting rod in the prior art is relatively complicated, it is sometimes inconvenient to use. However, most of the tube cores on the market are difficult to bend or have complex structures in the bent part, making it difficult to popularize in practice.

### SUMMARY

The purpose of the present disclosure is to overcome the shortcomings of the prior art, adapt to actual needs, and provide a new type of laryngoscope tube core.

To achieve the purpose of the disclosure, the technical solution adopted by the present disclosure is: a new type of laryngoscope tube core, including a smart connector shell, a tube core and an endoscope arranged at the end of the tube core. The smart connector shell includes an upper cover and a lower cover and the smart connector shell is provided with a circuit board. The circuit board is provided with a single-chip microcomputer. One end of the smart connector shell is provided with a MINI USB plug, and the MINI USB plug passes through the smart connector shell and is connected to the circuit board. The tube core is inserted into and fixed to the smart connector shell, and the end of the tube core inserted into the smart connector shell is connected to the circuit board. The tube core is provided with a layer of coil, and the coil is formed by flattening and winding a piece of plastic spring steel. The coil is covered with a layer of food-grade plastic tube. The end of the tube core protruding from one end of the smart connector shell is provided with an endoscope, and the endoscope is connected to the circuit board by a wire arranged in the tube core.

Preferably, the endoscope includes a steel sleeve a camera arranged in the steel sleeve, and an anti-fog lens is provided at the end of the steel sleeve; the camera is arranged inside a side of the steel sleeve with the anti-fog lens.

Preferably, the end of the tube core inserted into the end of the smart connector shell is provided with a boss for positioning, and the boss is arranged inside the smart connector shell.

The smart connector shell is provided with a circuit slot and a fixing slot; the boss matches the fixing slot; the circuit board is arranged in the circuit slot.

The coil is made of spring steel; the tube core is made of brass.

At least a pair of buckle/slot is provided on the upper cover, and the lower cover is provided with a pair of slot/buckle matching the buckle/slot.

A bolt hole is provided on one side of the lower cover, and a bolt is provided in the bolt hole; the boss is fixedly connected to the smart connector shell by the bolt provided in the bolt hole.

The beneficial effects of the present disclosure are as follows.

The tube core can be bent and shaped at will, convenient for intubation, and one-time used to avoid cross-infection;

The images can be real-time wirelessly visually transmitted and the position is accurate;

The tube core and the handle are connected by MINI USB, which is stable, reliable and durable.

### BRIEF DESCRIPTION OF THE DRAWINGS

Hereinafter, the present disclosure will be further explained with reference to the drawings and embodiments.

FIG. 1, FIG. 2, and FIG. 3 are exploded views of the present disclosure;

FIG. 4 is a partial enlarged schematic diagram of FIG. 3;

FIG. 5 and FIG. 6 are schematic diagrams of the coil of the present disclosure.

### IN THE DRAWINGS

1 smart connector shell; 10 upper cover; 101 buckle;
11 lower cover; 201 slot; 202 bolt hole; 203 bolt;
3 circuit board; 301 MINI USB plug; 302 single-chip microcomputer;
4 tube core; 401 coil; 402 food-grade plastic tube; 403 wire;
5 boss;
6 endoscope; 601 steel sleeve; 602 camera;
7 anti-fog lens;
8 fixing slot;
9 circuit slot.

### DETAILED DESCRIPTION OF EMBODIMENTS

In the following, the present disclosure will be further described in conjunction with the drawings and embodiments.

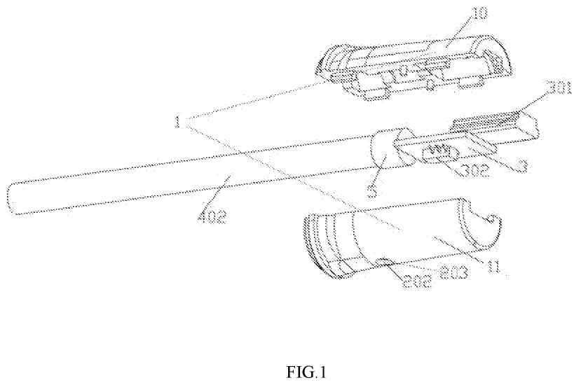

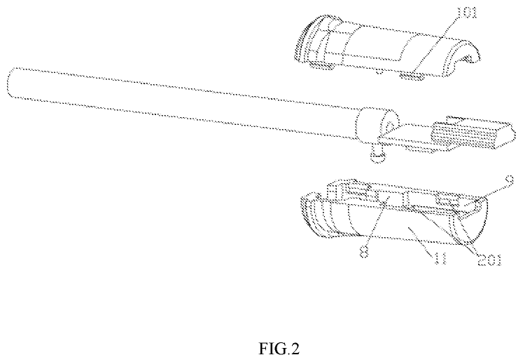

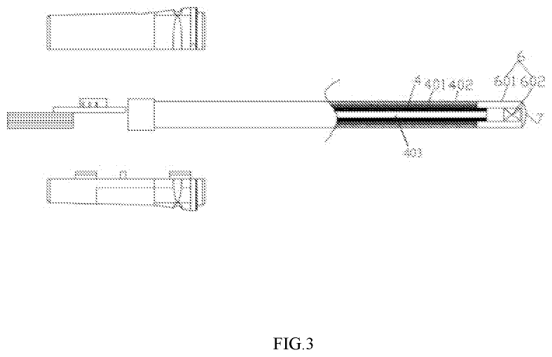

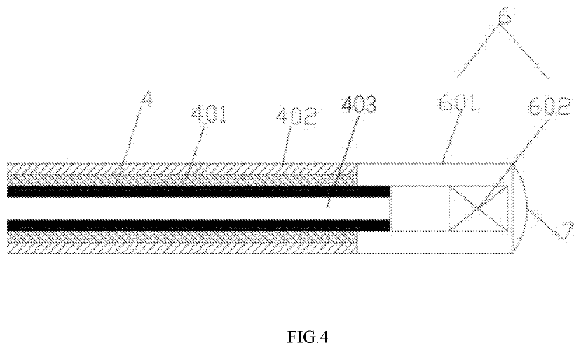

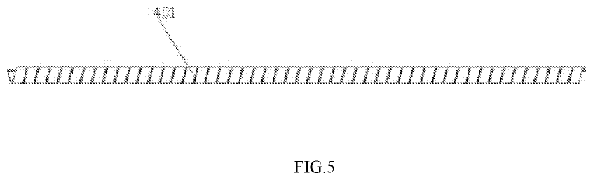

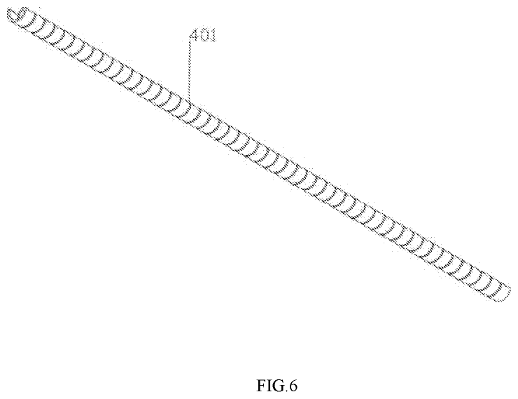

FIGS. 1-6 are taken for reference.

The present disclosure is a new type of laryngoscope tube core, including a smart connector shell 1, a tube core 4 and an endoscope 6 arranged at the end of the tube core 4. The smart connector shell 1 includes an upper cover 10 and a lower cover 11 and the smart connector shell 1 is provided with a circuit board 3. The circuit board 3 is provided with a single-chip microcomputer 302. One end of the smart connector shell 1 is provided with a MINI USB plug 301, and the MINI USB plug 301 passes through the smart connector shell 1 and is connected to the circuit board 3. The tube core 4 is inserted into and fixed to the smart connector shell 1, and the end of the tube core 4 inserted into the smart connector shell 1 is connected to the circuit board 3. The tube core 4 is provided with a layer of coil 401, and the coil 401 is formed by flattening and winding a piece of plastic spring steel. The coil 401 is covered with a layer of food-grade plastic tube 402. The end of the tube core 4 protruding from one end of the smart connector shell 1 is provided with an endoscope 6, and the endoscope 6 is connected to the circuit board 3 by a wire 403 arranged in the tube core 4. In actual use, the tube core 4 can be easily bent to reach the state required by medical personnel.

The endoscope 6 includes a steel sleeve 601, a camera 602 arranged in the steel sleeve 601, and an anti-fog lens 7 is provided at the end of the steel sleeve 601. The camera 602 is arranged inside a side of the steel sleeve 601 with the anti-fog lens 7.

The end of the tube core 4 inserted into the end of the smart connector shell 1 is provided with a boss 5 for positioning, and the boss 5 is arranged inside the smart connector shell 1.

The smart connector shell 1 is provided with a circuit slot 9 and a fixing slot 8. The boss 5 matches the fixing slot 8; the circuit board 3 is arranged in the circuit slot 9.

The coil 401 is made of spring steel. The tube core 4 is made of brass.

At least a pair of buckle 101/slot is provided on the upper cover 1, and the lower cover 2 is provided with a pair of slot 201/buckle matching the buckle 101/slot.

a bolt hole 202 is provided on one side of the lower cover 11, and a bolt 203 is provided in the bolt hole 202; the boss 5 is fixedly connected to the smart connector shell 1 by the bolt 203 provided in the bolt hole 202.

Described above are merely illustrative of the disclosure to enable those skilled in the art to implement or use the disclosure, and are not intended to limit the invention. It should be understood that any modifications, replacements or changes made by those skilled in the art without departing from the spirit of the disclosure should fall within the scope of the disclosure.

What is claimed is:

1. A new type of laryngoscope tube core, comprising:
a smart connector shell (1), a tube core (4) and an endoscope (6) arranged at the end of the tube core (4);
wherein the smart connector shell (1) comprises an upper cover (10) and a lower cover (11) and the smart connector shell (1) is provided with a circuit board (3); the circuit board (3) is provided with a single-chip microcomputer (302); one end of the smart connector shell (1) is provided with a MINI USB plug (301), and the MINI USB plug (301) passes through the smart connector shell (1) and is connected to the circuit board (3); the tube core (4) is inserted into and fixed to the smart connector shell (1), and the end of the tube core (4) inserted into the smart connector shell (1) is connected to the circuit board (3); the tube core (4) is provided with a layer of coil (401), and the coil (401) is formed by flattening and winding a piece of plastic spring steel; the coil (401) is covered with a layer of food-grade plastic tube (402); the end of the tube core (4) protruding from one end of the smart connector shell (1) is provided with an endoscope (6), and the endoscope (6) is connected to the circuit board (3) by a wire (403) arranged in the tube core (4).

2. The new type of laryngoscope tube core of claim 1, wherein the endoscope (6) comprises a steel sleeve (601), a camera (602) arranged in the steel sleeve (601), and an anti-fog lens (7) is provided at the end of the steel sleeve (601); the camera (602) is arranged inside a side of the steel sleeve (601) with the anti-fog lens (7).

3. The new type of laryngoscope tube core of claim 1, wherein the end of the tube core (4) inserted into the end of the smart connector shell (1) is provided with a boss (5) for positioning, and the boss (5) is arranged inside the smart connector shell (1).

4. The new type of laryngoscope tube core of claim 3, wherein the smart connector shell (1) is provided with a circuit slot (9) and a fixing slot (8); the boss (5) matches the fixing slot (8); the circuit board (3) is arranged in the circuit slot (9).

5. The new type of laryngoscope tube core of claim 1, wherein the coil (401) is made of spring steel; the tube core (4) is made of brass.

6. The new type of laryngoscope tube core of claim 1, wherein at least a pair of buckle (101)/slot is provided on the upper cover (1), and the lower cover (2) is provided with a pair of slot (201)/buckle matching the buckle (101)/slot.

7. The new type of laryngoscope tube core of claim 4, wherein a bolt hole (202) is provided on one side of the lower cover (11), and a bolt (203) is provided in the bolt hole (202); the boss (5) is fixedly connected to the smart connector shell (1) by the bolt (203) provided in the bolt hole (202).
